(19) United States
(12) Patent Application Publication
(10) Pub. No.: US 2012/0265237 A1
(43) Pub. Date: Oct. 18, 2012

(54) DETACHABLE EMBOLIZATION COIL SLEEVE

(76) Inventor: Kathryn R. EVERT, Bloomington, IN (US)

(21) Appl. No.: 13/086,846

(22) Filed: Apr. 14, 2011

(51) Int. Cl. A61F 2/01

(52) U.S. Cl. 606/200

(57) ABSTRACT

The present invention provides a delivery system for deploying an embolization coil. The delivery system comprises a delivery tube, a flexible sleeve extending distally from the delivery tube, and a mandril slidably disposed in the lumens of the delivery tube and the flexible sleeve. The present invention also provides an embolization coil delivery assembly comprising the delivery system of the present invention and an embolization coil. A proximal portion of the embolization coil is frictionally engaged by the flexible sleeve of the delivery system. The present invention also provides a method for positioning and deploying an embolization coil at a deployment site in a patient's vasculature using the delivery assembly of the present invention.

### BACKGROUND

The present invention relates to medical devices. More particularly, the present invention relates to a delivery system for deploying an embolization coil and a method for positioning and deploying an embolization coil at a deployment site in a patient's vasculature.

Embolization coils and microcoils are used in the peripheral, cardiac, and neurological vasculature to occlude blood vessels for a variety of clinical goals. Two basic delivery methods have been developed for the delivery and deployment of a coil at a desired deployment site in a patient's vasculature. In both methods, a catheter is advanced through the patient's vasculature until the distal end of the catheter reaches the deployment site.

In the basic pushable coil delivery method, the coil is advanced through the lumen of the catheter and out the distal end of the catheter at the deployment site using a pusher wire. The pusher wire does not attach to the coil. While these pushable coil methods offer simplicity in use, they often provide insufficient control of the coil during deployment.

In detachable coil delivery methods, the pusher wire attaches to the coil. After deployment of the coil, a medical practitioner detaches the pusher wire from the coil before removing the delivery system from the patient's vasculature. Detachable coils are particularly useful when the anatomy of the patient's vasculature is distal and tortuous and when very precise placement of the coil is needed. However, detachment of the pusher wire from the coil is often difficult.

The present invention generally provides a delivery system, a delivery assembly, and a method for deploying an embolization coil at a deployment site in a patient's vasculature. Embodiments of the present invention provide enhanced control of the embolization coil during the positioning and deployment of the coil. These embodiments also provide a simpler and more reliable means of coil detachment and deployment than existing detachable coil delivery systems.

In one embodiment, the present invention provides a delivery system for deploying an embolization coil having a proximal portion. The delivery system comprises a delivery tube having a first end and a second end. The delivery tube has a first lumen formed through the first end and the second end. The first lumen has a first lumen diameter. The delivery system further comprises a flexible sleeve having a third end and a fourth end. The flexible sleeve extends distally from the second end of the delivery tube to the fourth end of the flexible sleeve. The flexible sleeve has a second lumen formed through the third end and the fourth end and in fluid communication with the first lumen. The second lumen has a second lumen diameter. The flexible sleeve is configured to frictionally engage the proximal portion of the embolization coil in the second lumen. The delivery system further comprises a mandril slidably disposed within the first lumen and the second lumen to push the proximal portion of the embolization coil out of the second lumen to deploy the embolization coil.

In another embodiment, the present invention provides an embolization coil delivery assembly. The delivery assembly comprises a delivery tube having a first end and a second end. The delivery tube has a first lumen formed through the first end and the second end. The first lumen has a first lumen diameter. The delivery assembly further comprises a flexible sleeve having a third end and a fourth end. The flexible sleeve extends distally from the second end of the delivery tube to the fourth end of the flexible sleeve. The flexible sleeve has a second lumen formed through the third end and the fourth end and in fluid communication with the first lumen. The second lumen has a second lumen diameter. The delivery assembly further comprises an embolization coil having a proximal portion and a distal portion. The proximal portion of the embolization coil is disposed within the second lumen of the flexible sleeve. The flexible sleeve frictionally engages the proximal portion of the embolization coil. The delivery assembly further comprises a mandril slidably disposed within the first lumen and the second lumen to push the proximal portion of the embolization coil out of the second lumen to deploy the embolization coil.

In yet another embodiment, the present invention provides a method for positioning and deploying an embolization coil at a deployment site in a patient's vasculature. The method comprises percutaneously inserting a catheter into the patient's vasculature. The cathether has a proximal end and a distal end. The catheter has a catheter lumen formed through the proximal end and the distal end. The method further comprises advancing the catheter through the patient's vasculature until the distal end of the catheter is disposed at the deployment site, and the proximal end of the catheter is disposed outside of the patient's vasculature. The method further comprises inserting an embolization coil delivery assembly constructed in accordance with the present invention into the catheter lumen through the proximal end of the catheter. The method further comprises positioning the embolization coil at the deployment site. The method further comprises advancing the mandril distally through the first lumen and the second lumen to push the proximal portion of the embolization coil out of the second lumen to deploy the embolization coil.

Further objects, features, and advantages of the present invention will become apparent from consideration of the following description and the appended claims when taken in connection with the accompanying drawings.

### BRIEF DESCRIPTION OF THE DRAWINGS

FIG. 1 is a cross-sectional view of a delivery system for deploying an embolization coil in accordance with one embodiment of the present invention;

FIG. 2a is a cross-sectional view of a delivery system for deploying an embolization coil in accordance with a second embodiment of the present invention;

FIG. 2b is a cross-sectional view of a delivery system for deploying an embolization coil in accordance with a third embodiment of the present invention;

FIG. 2c is a cross-sectional view of a delivery system for deploying an embolization coil in accordance with a fourth embodiment of the present invention;

FIG. 3 is a cross-sectional view of an embolization coil delivery assembly in accordance with another embodiment of the present invention;

FIG. 4 is a flowchart depicting a method for positioning and deploying an embolization coil at a deployment site in a patient's vasculature in accordance with yet another embodiment of the present invention;

FIG. 5 is an environmental break-away view of a catheter extending to a deployment site in a patient's vasulature;

FIG. 6 is an environmental break-away view of the delivery assembly of FIG. 3 disposed in a catheter lumen near a deployment site in a patient's vasulature;

FIG. 7 is another environmental break-away view of the delivery assembly of FIG. 3 disposed in a catheter lumen at a deployment site in a patient's vasulature;

FIG. 8 is yet another environmental break-away view of the delivery assembly of FIG. 3 in which an embolization coil is partially deployed at a deployment site in a patient's vasulature; and

FIG. 9 is still another environmental break-away view of the delivery assembly of FIG. 3 in which an embolization coil is fully deployed at a deployment site in a patient's vasulature.

### DETAILED DESCRIPTION

The present invention generally provides a delivery system, a delivery assembly, and a method for deploying an embolization coil at a deployment site in a patient's vasculature. Embodiments of the present invention provide enhanced control of the embolization coil during the positioning and deployment of the coil. These embodiments also provide a simpler and more reliable means of coil detachment and deployment than existing detachable coil delivery systems.

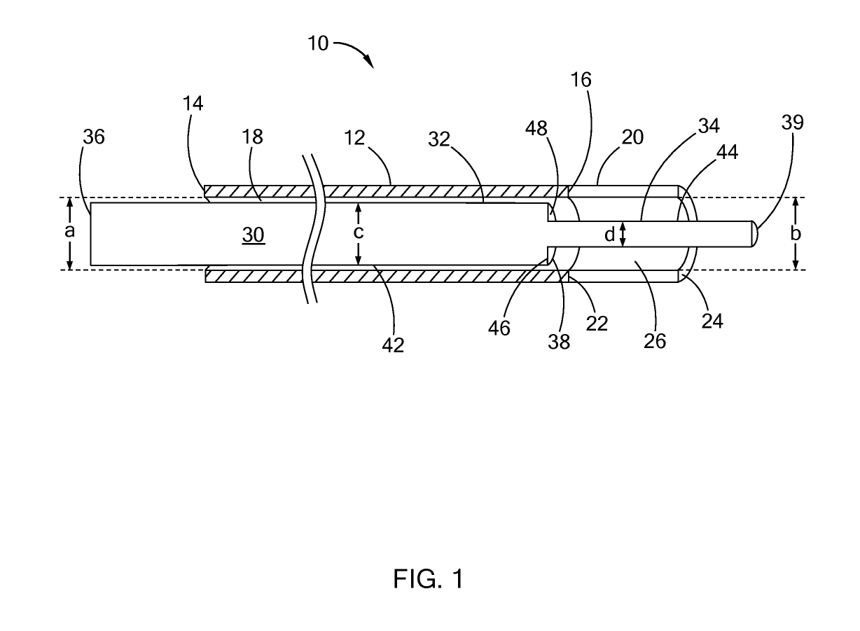

FIG. 1 illustrates a delivery system 10 for deploying an embolization coil in accordance with one embodiment of the present invention. The delivery system 10 comprises a delivery tube 12, a flexible sleeve 20, and a mandril 30.

As shown in FIG. 1, the delivery tube 12 has a proximal first end 14 and a distal second end 16. The delivery tube 12 has a substantially cylindrical inner surface defining a first lumen 18 formed through the first end 14 and the second end 16 of the delivery tube 12.

The delivery tube 12 may be constructed from any suitable material that will provide sufficient flexibility and resistance to kinking. For example, the delivery tube 12 may be constructed from stainless steel, platinum, nitinol, nylon, braid reinforced nylon, or any other suitable material. In some embodiments, the delivery tube 12 may be constructed as a coiled wire guide.

The flexible sleeve 20 has a proximal third end 22 and a distal fourth end 24. The flexible sleeve 20 extends distally from the second end 16 of the delivery tube 12 to the fourth end 24 of the flexible sleeve 20. In the embodiment shown in FIG. 1, the flexible sleeve has a substantially cylindrical inner surface defining a second lumen 26 formed through the third end 22 and the fourth end 24 of the flexible sleeve 20. The second lumen 26 is in fluid communication with the first lumen 18 of the delivery tube 12. The flexible sleeve 20 is configured to frictionally engage a proximal portion of an embolization coil in the second lumen 26. The length of the flexible sleeve 20 is selected to achieve a frictional engagement of sufficient strength between the flexible sleeve 20 and the proximal portion of the embolization coil. Preferably, the distance between the second end 16 of the delivery tube 12 and the fourth end 24 of the flexible sleeve 20 is between about 2 mm and about 6 mm, more preferably between about 3 mm and about 5 mm, and most preferably between about 3.5 mm and about 4.5 mm.

The flexible sleeve 20 may be constructed from a flexible polymer, such as nylon or polytetrafluoroethylene (PTFE), or any other suitable material. Preferably, the flexible sleeve 20 is constructed from a flexible polymer. In some embodiments, the flexible sleeve 20 may be constructed from a dissolvable agent, such as poly-lactic-co-glycolic acid (PLGA), or any other suitable dissolvable agent. The flexible sleeve 20 may also include a radiopacifier, such as barium sulfate, or any other suitable radiopacifier.

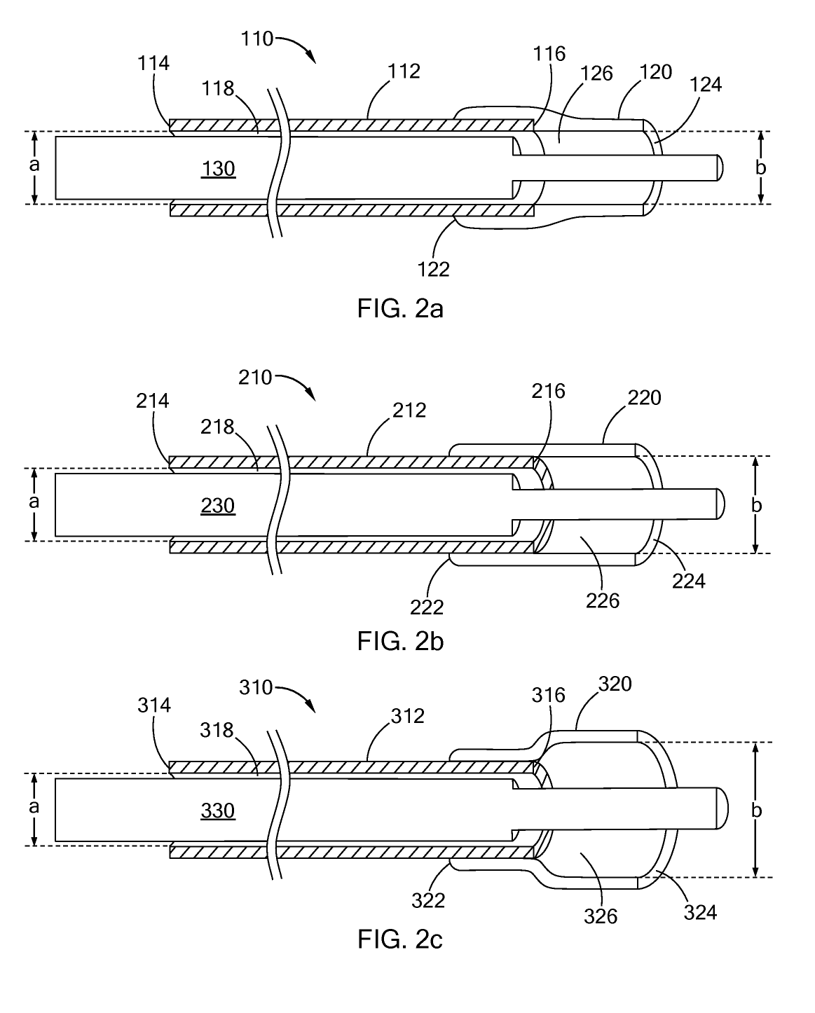

The flexible sleeve 20 is fixedly attached to the delivery tube 12. In the embodiment shown in FIG. 1, the third end 22 of the flexible sleeve 20 abutts against the second end 16 of the delivery tube 12 to define an abutting joint. In other embodiments, such as those shown in FIGS. 2a-2c, the flexible sleeve 120, 220, or 320 may overlap a distal portion of the delivery tube 112, 212, or 312 to define a lap joint. In these embodiments, the third end 122, 222, or 322 of the flexible sleeve 120, 220, or 320 is disposed proximally to the second end 116, 216, or 316 of the delivery tube 112, or 212, or 312. In the embodiments shown in FIGS. 2a-2c, the amount of overlap between the flexible sleeve 120, 220, or 320 and the delivery tube 112, 212, or 312, i.e., the distance between the third end 122, 222, or 322 of the flexible sleeve 120, 220, or 320 and the second end 116, 216, or 316 of the delivery tube 112, 212, or 312, is selected to provide a sufficiently strong attachment between the flexible sleeve 120, 220, or 320 and the delivery tube 112, 212, or 312. The flexible sleeve 20 may also be attached to the delivery tube 12 by any other means of joinery known to those having ordinary skill in the relevant art. Moreover, the attachment of the flexible sleeve 20, 120, 220, or 320 to the delivery tube 12, 112, 212, or 312 may be accomplished by adhesive bonding, thermal bonding, mechanical clamping, or any other suitable means of attachment.

As shown in FIGS. 1-2c, the first lumen 18 and the second lumen 26 have a first lumen diameter a and a second lumen diameter b, respectively. The first lumen diameter a is selected based on the desired properties of the delivery tube 12 for a particular application. A larger first lumen diameter a may be selected if a more rigid delivery tube 12 is desired. Conversely, a smaller first lumen diameter a may be selected if a more flexible delivery tube 12 is desired. Preferably, the first lumen diameter a is between about 0.015 and 0.017 inches, more preferably about 0.016 inches, or between about 0.023 and 0.027 inches, more preferably about 0.025 inches.

Similarly, the second lumen diameter b is selected based on the size of embolization coil to be used for a particular application. The second lumen diameter b is preferably selected to be slightly smaller than the outer diameter of the embolization coil to be deployed using the delivery system, such that the flexible sleeve 20 forms a tight friction fit with the embolization coil. Preferably, the second lumen diameter b is between about 0.014 and 0.016 inches, more preferably about 0.015 inches, or between about 0.021 and 0.023 inches, more preferably about 0.022 inches.

Depending on the desired properties of the delivery tube 12 and the size of embolization coil to be used for a particular application, as described above, the second lumen diameter b may be substantially equal to or greater than the first lumen diameter a. In the embodiments shown in FIGS. 1 and 2a, the second lumen diameter b of the second lumens 26 and 126 is substantially equal to the first lumen diameter a of the first lumens 18 and 118. In the embodiments shown in FIGS. 2b and 2c, the second lumen diameters b of the second lumens 226 and 326 are greater than the first lumen diameters a of the first lumens 218 and 318. As shown in FIG. 2b, the second lumen diameter b of the second lumen 226 may be substantially equal to the outer diameter of the delivery tube 212. Alternatively, as shown in FIG. 2c, the flexible sleeve 320 may have a flared shape, such that the second lumen diameter b of the second lumen 326 is greater than the outer diameter of the delivery tube 312.

Referring again to FIG. 1, the mandril 30 is slidably disposed within the first lumen 18 and the second lumen 26 to push the proximal portion of the embolization coil out of the second lumen 26 to deploy the embolization coil. As shown in FIG. 1, the mandril 30 preferably has a proximal first portion 32 and a distal second portion 34. The first portion 32 of the mandril 30 has a proximal fifth end 36, a distal sixth end 38, and a first substantially cylindrical surface 42. The distance between the fifth end 36 and the sixth end 38 is greater than the distance between the first end 14 of the delivery tube 12 and the fourth end 24 of the flexible sleeve 20 so that the mandril 30 can be used like a plunger to push an embolization coil out of the delivery system 10. The second portion 34 of the mandril 30 extends distally from a proximal seventh end adjoining the sixth end 38 of the first portion 32 to a distal eighth end 39. The distance between the seventh end and the eighth end 39 of the second portion 34 of the mandril 30 is preferably between about 4 mm and about 8 mm, more preferably between about 5 mm and about 7 mm, and most preferably between about 5.5 mm and about 6.5 mm. The second portion 34 of the mandril 30 has a second substantially cylindrical surface 44. The first substantially cylindrical surface 42 and the second substantially cylindrical surface 44 preferably share a common longitudinal axis.

The mandril 30 may be constructed from a superelastic material (e.g., nitinol), a metal (e.g., stainless steel), or any other suitable material. Preferably, the mandril 30 is constructed from nitinol.

The first substantially cylindrical surface 42 and the second substantially cylindrical surface 44 have a first portion diameter c and a second portion diameter d, respectively. As shown in FIG. 1, the second portion diameter d is preferably smaller than the first portion diameter c. As shown in FIG. 1, and as noted above, the mandril 30 is slidably disposed within the first lumen 18 and the second lumen 26. Thus, the first portion diameter c is selected to be less than or equal to each of the first lumen diameter a and the second lumen diameter b. Preferably, the first portion diameter c is slightly smaller than the smaller of the first lumen diameter a and the second lumen diameter b.

The first portion diameter c and the second portion diameter d are also selected based on the size of embolization coil to be used for a particular application. The first portion diameter c is selected to be larger than the inner diameter of the embolization coil, and the second portion diameter d is selected to be smaller than the inner diameter of the embolization coil. Preferably, the first portion diameter c is between about 0.005 and 0.007 inches, more preferably about 0.006 inches, or between about 0.010 and 0.014 inches, more preferably about 0.012 inches. Preferably, the second portion diameter d is between about 0.003 and 0.005 inches, more preferably about 0.004 inches, or between about 0.006 and 0.010 inches, more preferably about 0.008 inches.

In a preferred embodiment, as shown in FIG. 1, the sixth end 38 of the first portion 32 defines a shoulder 46. The shoulder 46 has a shoulder surface 48. Preferably, the shoulder surface 48 is substantially orthogonal to the first substantially cylindrical surface 42 and the second substantially cylindrical surface 44. As used herein, the shoulder surface 48 shall be deemed to be “substantially orthogonal” to the first substantially cylindrical surface 42 and the second substantially cylindrical surface 44 if the shoulder surface 48 forms about a 70-110° angle, preferably about an 80-100° angle, with the surfaces 42 and 44.

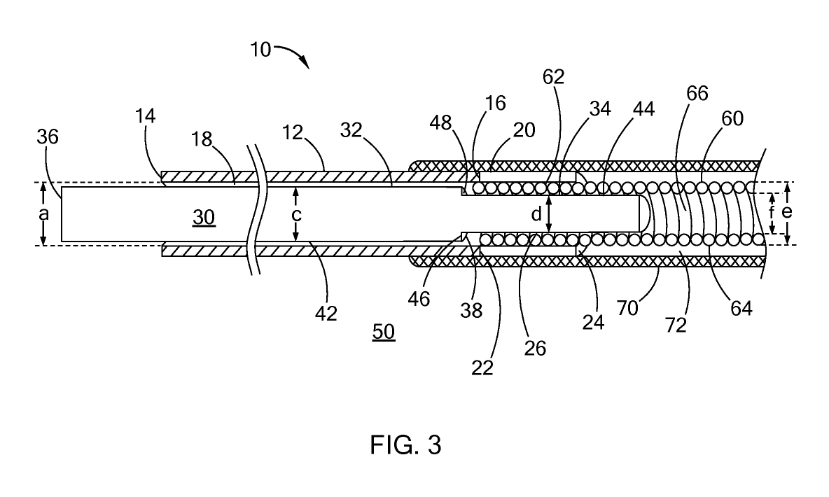

FIG. 3 illustrates an embolization coil delivery assembly 50 in accordance with another embodiment of the present invention. The delivery assembly 50 comprises the delivery system 10 and an embolization coil 60. The embolization coil 60 has a proximal end, a proximal portion 62, a distal portion 64, and a distal end. The embolization coil 60 also has a coil lumen 66 formed through the proximal and distal ends of the coil. The proximal portion 62 of the embolization coil 60 is disposed within the second lumen 26 of the flexible sleeve 20. The flexible sleeve 20 frictionally engages the proximal portion 62 of the embolization coil 60.

The embolization coil 60 has an outer diameter e and an inner diameter f. Preferably, the outer diameter e of the embolization coil 60 is between about 0.012 and 0.016 inches, more preferably between about 0.013 and 0.015 inches, or between about 0.016 and 0.026 inches, more preferably between about 0.019 and 0.023 inches. Preferably, the inner diameter f of the embolization coil 60 is between about 0.004 and 0.006 inches, more preferably about 0.005 inches, or between about 0.008 and 0.012 inches, more preferably about 0.010 inches. As shown in FIG. 3, and as discussed in more detail above, the second lumen diameter b is preferably selected to be slightly smaller than the outer diameter e of the embolization coil 60, such that the flexible sleeve 20 forms a tight friction fit with the proximal portion 62 of the embolization coil 60. In addition, the first portion diameter c of the mandril 30 is greater than the inner diameter f of the embolization coil 60, which is greater than the second portion diameter d of the mandril 30. So dimensioned, the second portion 34 of the mandril 30 can slide into the coil lumen 66, as shown in FIG. 3, but the first portion 32 of the mandril 30 cannot slide into the coil lumen 66.

The embolization coil 60 may be constructed from stainless steel wire, a superelastic material, cobalt-chromium-nickel-molybdenum-iron alloy, cobalt-chrome alloy, platinum or any other suitable material.

As shown in FIG. 3, the delivery assembly 50 may also include a shipping cannula 70. The shipping cannula 70 has a tubular construction and a third lumen 72. The embolization coil 60 and the flexible sleeve 20 are disposed within the third lumen 72 of the shipping cannula 70. The shipping cannula 70 constrains the embolization coil 60 in a substantially linear configuration prior to deployment. The shipping cannula may be constructed from a rigid metal or plastic, or any other suitable material.

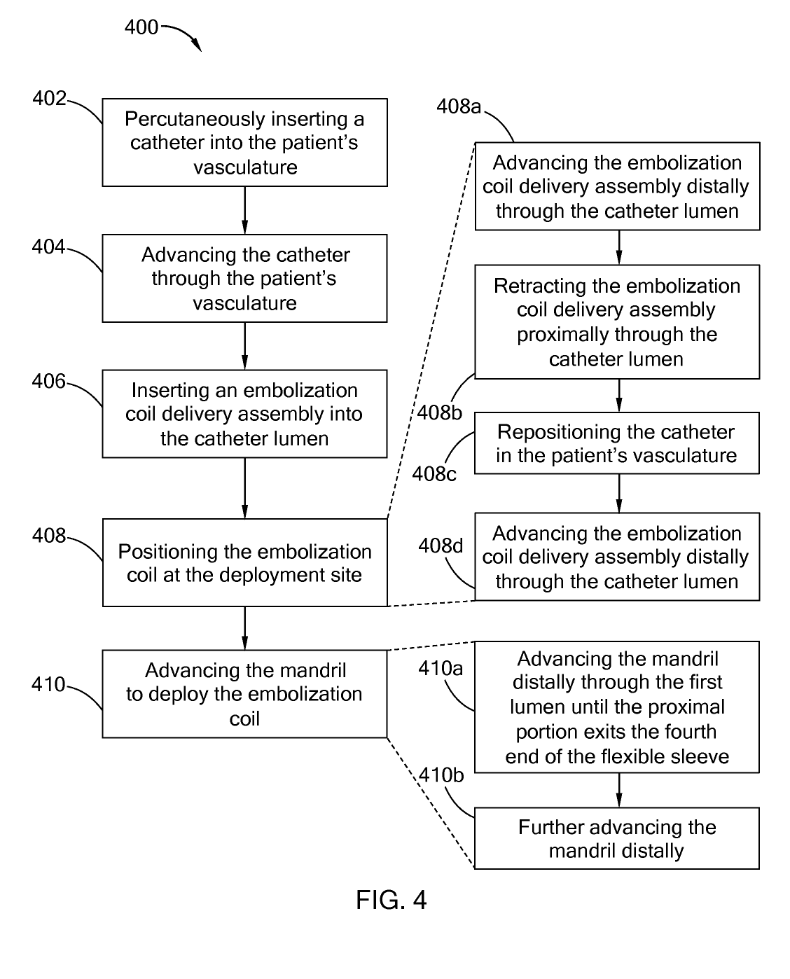

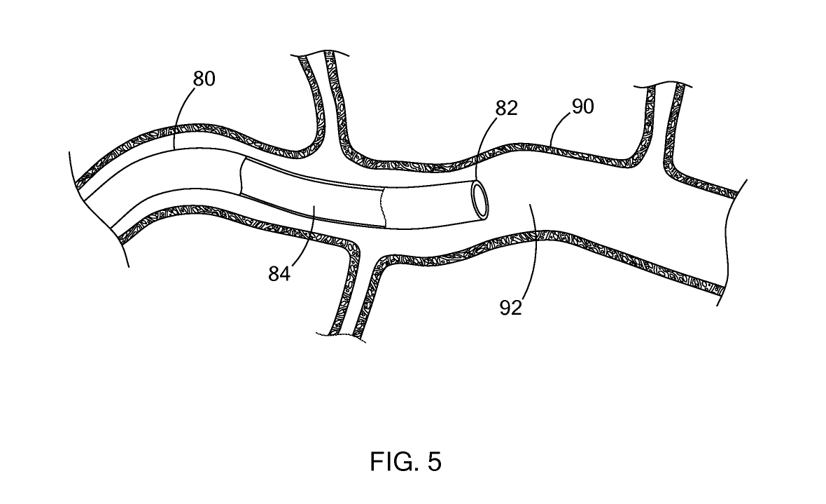

FIG. 4 illustrates a flow chart depicting a method 400 for positioning and deploying an embolization coil at a deployment site in a patient's vasculature, implementing the delivery assembly 50 described above, in accordance with yet another embodiment of the present invention. As indicated in box 402, and as illustrated in FIG. 5, the method 400 comprises percutaneously inserting a catheter 80 into the patient's vasculature 90. The catheter has a proximal end (not shown) and a distal end 82. The catheter 80 also has a catheter lumen 84 formed through the proximal end and the distal end 82 of the catheter 80. As indicated in box 404, the method 400 further comprises advancing the catheter 80 through the patient's vasculature 90 until the distal end 82 of the catheter 80 is disposed at the deployment site 92. As the catheter 80 is advanced through the vasculature 90, the proximal end of the catheter 80 remains outside of the patient's vasculature 90.

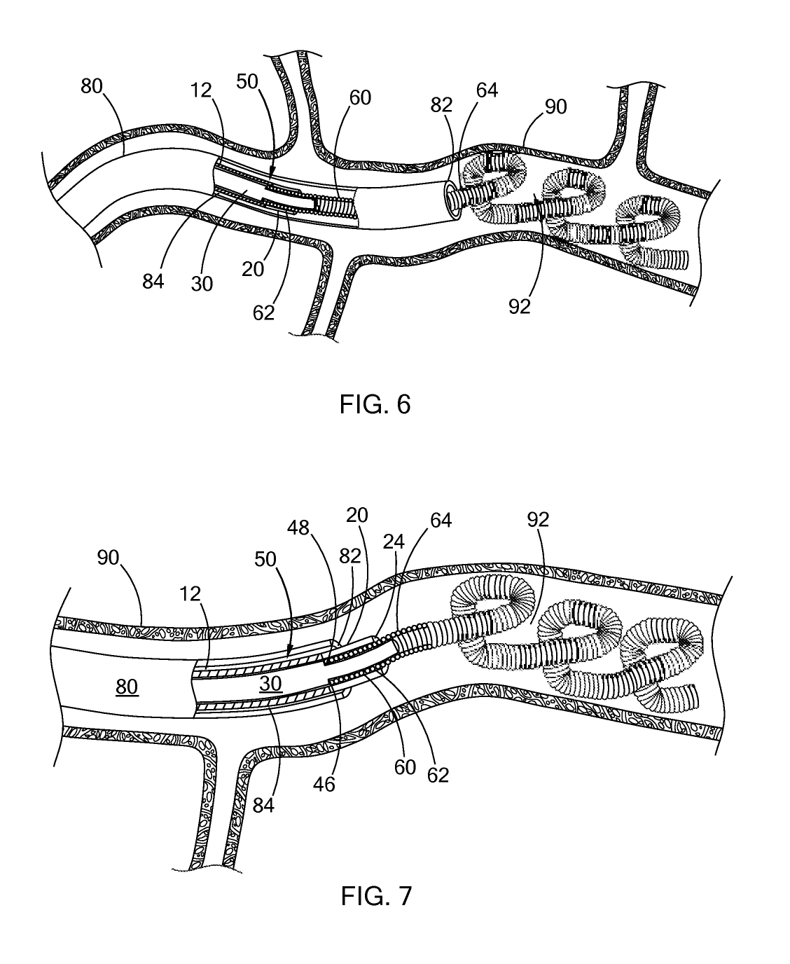

As indicated in box 406, and as illustrated in FIG. 6, the method 400 further comprises inserting the embolization coil delivery assembly 50 of the present invention into the catheter lumen 84 through the proximal end of the catheter 80. If the delivery assembly 50 includes a shipping cannula, the shipping cannula is placed in a hub at the proximal end of the catheter 80. The delivery system 50 is fed into the catheter lumen 84 through the third lumen of the shipping cannula. Thus, the distal portion 64 of the embolization coil 60 is fed into the catheter lumen 84, and the flexible sleeve 20 and the delivery tube 12 are fed through the third lumen of the shipping cannula to advance the distal portion 64 of the embolization coil in the catheter lumen 84. The catheter 80 constrains the distal portion 64 of the embolization coil 60 in a substantially linear configuration. FIG. 6 illustrates a cut-away in the wall of the catheter 80, revealing a cross-sectional view of the delivery assembly 50 in the catheter lumen 84.

As shown in FIG. 6, as the delivery assembly 50 is fed through the catheter lumen 84, the distal portion 64 of the embolization coil 60 eventually begins to exit the distal end 82 of the catheter 80 at the deployment site 92. As the distal portion 64 of the embolization coil 60 exits the catheter lumen 84, it coils up to occlude the patient's vasculature 90 at the deployment site 92.

As indicated in box 408, the method 400 further comprises positioning the embolization coil 60 at the deployment site 92. The step of positioning the embolization coil 60 may comprise the series of sub-steps indicated in boxes 408a-408d.

As indicated in box 408a, and as illustrated in FIG. 7, the step indicated in box 408 may comprise advancing the embolization coil delivery assembly 50 distally through the catheter lumen 84 by feeding more of the delivery tube 12 into the catheter lumen 84 until the fourth end 24 of the flexible sleeve 20 reaches the distal end 82 of the catheter 80. As the delivery assembly 50 is advanced through the catheter lumen 84, the distal portion 64 of the embolization coil 60 exits the catheter lumen 84 through the distal end 82 of the catheter 80 into the patient's vasculature 90 at the deployment site 92. As shown in FIG. 7, the distal portion 64 of the embolization coil 60 coils up to occlude the patient's vasculature 90 at the deployment site 92 after it exits the distal end 82 of the catheter 80. The proximal portion 62 of the embolization coil 60 is still disposed in the second lumen of the flexible sleeve 20.

If the flexible sleeve 20 includes a radiopacifier, a medical practitioner may monitor the location of the fourth end 24 of the flexible sleeve 20 by imaging to determine whether the positioning of the embolization coil 60 at the deployment site 92 is satisfactory. As indicated in box 408b, if the positioning of the embolization coil 60 is not satisfactory, the step indicated in box 408 may further comprise retracting the embolization coil delivery assembly 50 proximally through the catheter lumen 84 to pull the distal portion 64 of the embolization coil 60 back into the catheter lumen 84 through the distal end of the catheter 80. This retraction is accomplished by pulling part of the delivery tube 12 out of the catheter lumen 84 through the proximal end of the catheter 80. Such retraction is possible because of the friction fit between the flexible sleeve 20 and the proximal portion 62 of the embolization coil 60.

As indicated in box 408c, once the embolization coil 60 has been retracted into the catheter lumen 84, the practitioner may reposition the catheter 80 in the patient's vasculature 90 to more precisely place the distal end 82 of the catheter 80 at the deployment site 92. As indicated in box 408d, after properly positioning the distal end 82 of the catheter 80, the practitioner may once again advance the embolization coil delivery assembly 50 distally through the catheter lumen 84 until the fourth end 24 of the flexible sleeve 20 reaches the distal end 82 of the catheter 80, as shown in FIG. 7. As the fourth end 24 of the flexible sleeve 20 approaches the distal end 82 of the catheter 80, the distal portion 64 of the embolization coil 60 once again exits from the distal end 82 of the catheter 80 into the patient's vasculature 90 at the deployment site 92.

If the flexible sleeve 20 includes a radiopacifier, the practitioner may once again monitor the location of the fourth end 24 of the flexible sleeve 20 by imaging to determine whether the positioning of the embolization coil 60 at the deployment site 92 is satisfactory. If the positioning is not satisfactory, the steps indicated in boxes 408b-408d may be repeated as necessary to properly position the embolization coil 60 at the deployment site 92.

If the positioning is satisfactory, the practitioner may deploy the embolization coil 60, as indicated in box 410, by advancing the mandril 30 distally through the first lumen of the delivery tube 12 and the second lumen of the flexible sleeve 20. As the mandril 30 is advanced through the first and second lumens, the shoulder surface 48 of the mandril 30 contacts the proximal end of the embolization coil 60, pushing the proximal portion 62 of the embolization coil 60 out of the second lumen.

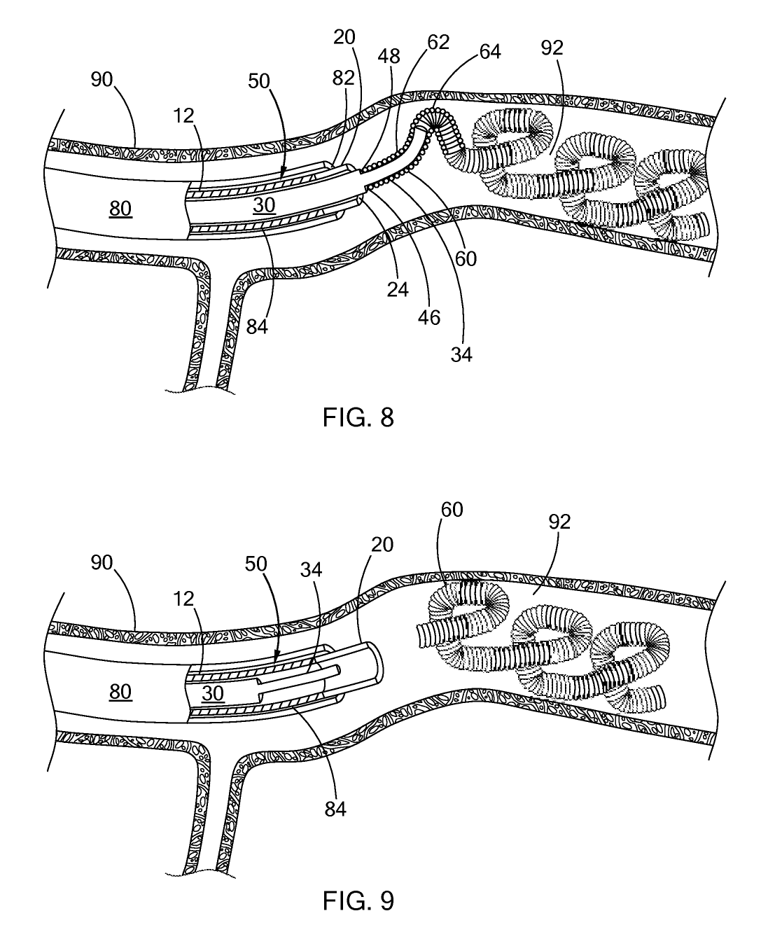

After the proximal portion 62 of the embolization coil 60 has been pushed out of the second lumen, as shown in FIG. 8, the second portion 34 of the mandril 30 remains in the coil lumen. As a result of this continued linkage between the mandril 30 and the embolization coil 60, the practitioner retains a measure of control over the embolization coil 60 even after the coil has been pushed out of the flexible sleeve 20. Thus, the step of advancing the mandril 30 to deploy the embolization coil 60 may comprise the series of sub-steps indicated in boxes 410a-410b.

As indicated in box 410a, the step indicated in box 410 may comprise advancing the mandril 30 distally through the first lumen and the second lumen until the proximal portion 62 of the embolization coil 60 exits the fourth end 24 of the flexible sleeve 20. As discussed above, the advancement of the mandril 30 causes the shoulder surface 48 to contact the proximal end of the embolization coil 60, pushing the proximal portion 62 of the embolization coil 60 out of the second lumen of the flexible sleeve 20.

As indicated in box 410b, the step indicated in box 410 may further comprise further advancing the mandril 30 distally through the first lumen and the second lumen to position the embolization coil 60 at the deployment site 92. As discussed above, even after the proximal portion 62 of the embolization coil 60 has been expelled from the flexible sleeve 20, as shown in FIG. 8, the practitioner retains a measure of control over the embolization coil 60 because the second portion 34 of the mandril 30 is disposed in the coil lumen. Thus, the practitioner may continue to push on the proximal end of the embolization coil 60 to position the coil. For example, the practitioner may push on the proximal end of the embolization coil 60 to tuck the proximal portion 62 of the coil into the occlusion formed by the distal portion 64 of the coil for more effective occlusion at the deployment site 92.

Once the practitioner is satisfied with the positioning of the embolization coil 60, the mandril 30 may be retracted proximally through the first and second lumens of the delivery tube 12 and the flexible sleeve 20. As shown in FIG. 9, the retraction of the mandril 30 withdraws the second portion 34 of the mandril 30 from the coil lumen. The delivery assembly 50 and the catheter 80 may then be removed from the patient's vasculature.

While the present invention has been described in terms of certain preferred embodiments, it will be understood that the invention is not limited to the disclosed embodiments, as those having skill in the art may make various modifications without departing from the scope of the following claims.

What is claimed is:

1. A delivery system for deploying an embolization coil having a proximal portion, the system comprising:
a delivery tube having a first end and a second end, the delivery tube having a first lumen formed through the first end and the second end, the first lumen having a first lumen diameter;
a flexible sleeve having a third end and a fourth end, the flexible sleeve extending distally from the second end of the delivery tube to the fourth end of the flexible sleeve, the flexible sleeve having a second lumen formed through the third end and the fourth end and in fluid communication with the first lumen, the second lumen having a second lumen diameter, the flexible sleeve being configured to frictionally engage the proximal portion of the embolization coil in the second lumen; and
a mandril slidably disposed within the first lumen and the second lumen to push the proximal portion of the embolization coil out of the second lumen to deploy the embolization coil.

2. The delivery system of claim 1, wherein the mandril has a first portion and a second portion, the first portion having a fifth end and a sixth end, the first portion having a first substantially cylindrical surface having a first portion diameter, the second portion extending distally from the sixth end of the first portion, the second portion having a second substantially cylindrical surface having a second portion diameter; wherein the first portion diameter is greater than the second portion diameter.

3. The delivery system of claim 2, wherein the sixth end defines a shoulder of the mandril, the shoulder having a shoulder surface.

4. The delivery system of claim 3, wherein the shoulder surface is substantially orthogonal to the first substantially cylindrical surface and the second substantially cylindrical surface.

5. The delivery system of claim 1, wherein the flexible sleeve is constructed from a flexible polymer.

6. The delivery system of claim 1, wherein the mandril is constructed from nitinol.

7. The delivery system of claim 1, wherein the second lumen diameter is greater than the first lumen diameter.

8. An embolization coil delivery assembly, the delivery assembly comprising:
a delivery tube having a first end and a second end, the delivery tube having a first lumen formed through the first end and the second end, the first lumen having a first lumen diameter;
a flexible sleeve having a third end and a fourth end, the flexible sleeve extending distally from the second end of the delivery tube to the fourth end of the flexible sleeve, the flexible sleeve having a second lumen formed through the third end and the fourth end and in fluid communication with the first lumen, the second lumen having a second lumen diameter;
an embolization coil having a proximal portion and a distal portion, the proximal portion of the embolization coil being disposed within the second lumen of the flexible sleeve, the flexible sleeve frictionally engaging the proximal portion of the embolization coil; and
a mandril slidably disposed within the first lumen and the second lumen to push the proximal portion of the embolization coil out of the second lumen to deploy the embolization coil.

9. The delivery assembly of claim 8, wherein the mandril has a first portion and a second portion, the first portion having a fifth end and a sixth end, the first portion having a first substantially cylindrical surface having a first portion diameter, the second portion extending distally from the sixth end of the first portion, the second portion having a second substantially cylindrical surface having a second portion diameter; wherein the first portion diameter is greater than the second portion diameter.

10. The delivery assembly of claim 9, wherein the sixth end defines a shoulder of the mandril, the shoulder having a shoulder surface.

11. The delivery assembly of claim 10, wherein the shoulder surface is substantially orthogonal to the first substantially cylindrical surface and the second substantially cylindrical surface.

12. The delivery assembly of claim 10, wherein the embolization coil has an inner diameter greater than the second portion diameter, the first portion diameter being greater than the inner diameter.

13. The delivery assembly of claim 8, wherein the flexible sleeve is constructed from a flexible polymer.

14. The delivery assembly of claim 8, wherein the mandril is constructed from nitinol.

15. The delivery assembly of claim 8, wherein the second lumen diameter is greater than the first lumen diameter.

16. The delivery assembly of claim 8, further comprising a shipping cannula having a third lumen, the embolization coil and the flexible sleeve being disposed within the third lumen of the shipping cannula.

17. A method for positioning and deploying an embolization coil at a deployment site in a patient's vasculature, the method comprising the steps of:
percutaneously inserting a catheter into the patient's vasculature, the cathether having a proximal end and a distal end, the catheter having a catheter lumen formed through the proximal end and the distal end;
advancing the catheter through the patient's vasculature until the distal end of the catheter is disposed at the deployment site, the proximal end of the catheter being disposed outside of the patient's vasculature;
inserting an embolization coil delivery assembly into the catheter lumen through the proximal end of the catheter, the delivery assembly comprising:
a delivery tube having a first end and a second end, the delivery tube having a first lumen formed through the first end and the second end, the first lumen having a first lumen diameter;
a flexible sleeve having a third end and a fourth end, the flexible sleeve extending distally from the second end of the delivery tube to the fourth end of the flexible sleeve, the flexible sleeve having a second lumen formed through the third end and the fourth end and in fluid communication with the first lumen, the second lumen having a second lumen diameter;
an embolization coil having a proximal portion and a distal portion, the proximal portion of the embolization coil being disposed within the second lumen of the flexible sleeve, the flexible sleeve frictionally engaging the proximal portion of the embolization coil; and
a mandril slidably disposed within the first lumen and the second lumen;
positioning the embolization coil at the deployment site; and
advancing the mandril distally through the first lumen and the second lumen to push the proximal portion of the embolization coil out of the second lumen to deploy the embolization coil.

18. The method of claim 17, wherein positioning the embolization coil comprises:
advancing the embolization coil delivery assembly distally through the catheter lumen until the fourth end of the flexible sleeve reaches the distal end of the catheter to exit the distal portion of the embolization coil from the distal end of the catheter into the patient's vasculature at the deployment site;
retracting the embolization coil delivery assembly proximally through the catheter lumen to pull the embolization coil back into the catheter lumen;
repositioning the catheter in the patient's vasculature; and
advancing the embolization coil delivery assembly distally through the catheter lumen until the fourth end of the flexible sleeve reaches the distal end of the catheter to exit the distal portion of the embolization coil from the distal end of the catheter into the patient's vasculature at the deployment site.

19. The method of claim 18, wherein advancing the mandril comprises:
advancing the mandril distally through the first lumen and the second lumen until the proximal portion of the embolization coil exits the fourth end of the flexible sleeve; and
further advancing the mandril distally through the first lumen and the second lumen to position the embolization coil at the deployment site.
